(19) United States
(12) Patent Application Publication
(10) Pub. No.: US 2024/0131567 A1
(43) Pub. Date: Apr. 25, 2024

(54) Tank mix adjuvant system for spore forming and nonspore forming bacteria for residue breakdown

(71) Applicant: Meristem Crop Performance Group, LLC, Powell, OH (US)

(72) Inventors: Lucas Loveless, Chatham, IL (US); Larry Fiene, Prairie Du Sac, WI (US); Joe Gednalske, River Falls, WI (US); Brian Haschemeyer, Athens, IL (US)

(21) Appl. No.: 18/391,226

(22) Filed: Dec. 20, 2023

Related U.S. Application Data
(63) Continuation-in-part of application No. 17/696,381, filed on Mar. 16, 2022.
(60) Provisional application No. 63/434,376, filed on Dec. 21, 2022.
(60) Provisional application No. 63/279,343, filed on Nov. 15, 2021.

(51) Int. Cl. B09B 3/60; C12N 1/20

(52) U.S. Cl. CPC B09B 3/60; C12N 1/20; B09B 2101/85

(57) ABSTRACT

A composition and method for application on plant stubble or plant residue left on a field after harvest wherein the composition comprises a blend of water, ammonium sulfate, anti-foam and alkyl polysaccharide in an approximate range of 10 to 30% alkyl polysaccharide in 0.1 to 3.4 lbs. of ammonium sulfate per 1 gallon of the blend and a bacteria spore blend or a blend of non-spore forming bacteria comprising bacteria suitable for degrading plant residue.

### CROSS-REFERENCE TO RELATED APPLICATIONS

The present application claims priority to and the benefit of U.S. provisional application Ser. No. 63/434,376, filed on Dec. 21, 2022, and is a continuation in part of U.S. nonprovisional application Ser. No. 17/696,381, filed on Mar. 16, 2022 which claims priority to and the benefit of U.S. provisional application Ser. No. 63/279,343, filed on Nov. 15, 2021, the contents of both which are hereby incorporated by reference in their entirety.

### BACKGROUND

This disclosure relates to the use of bacteria, both spore forming and non-spore forming, with an adjuvant to reduce cellulosic residue and to increase plant nutrients.

No-till farming, sometimes known as direct drilling, is an agricultural technique for growing crops without disturbing the soil prior to planting seed. In short, when crops are harvested and the stalks or stems of the agricultural plant are cut, stubble and other plant residue from the cut plants remain on the soil. Farmers using cover crops also have excess residue to deal with. Even using farming techniques that plow under stubble and plant residue, some stubble and plant residue may be left on the soil surface and extend upward through the soil surface. This stubble and plant residue results in an obstacle and on occasion a barrier for proper seeding when no till or direct drilling technique is used in the following planting season. If the soil was barren, the seeds would be planted at the proper depth. However, if the field has plant residue or stubble, the machinery that presents the seed to the soil and then pushes the seed into the soil rides on the surface of the stubble or plant residue resulting in the seed not being pushed into the soil sufficiently for proper germination or the seed is even left lying on the stubble or crop residue resulting in no germination. There have been attempts to reduce the stubble, primarily, the height of the stubble. For example, customized machinery can cut the stubble at a lower level. However, enough stubble is still left standing which continues the obstruction problem to the machinery that plants the seed, of not planting the seed properly for germination. In certain geographical areas, farmers have been known to burn the stubble. However, the disadvantages of burning are a loss of nutrients, a deleterious impact on soil microbes, reduction in soil structure, and increase in erosion and possible increase in soil acidity overtime.

Plant residue also occurs in non-agricultural settings such as grass planted for residential or commercials uses in golf courses, outdoor stadiums and similar commercial applications. Such natural turf requires constant cutting to ensure grass thickness and aesthetic pleasantness.

Constant cutting produces thatch which in some situations may become a tightly intermingled layer of living and dead stems, leaves and roots that accumulate over time due to the constant cutting of the living grass. Too great of an accumulation of thatch will result in a lawn or sports field having an unpleasant appearance.

Presently, there are microbial compositions that are designed to reduce residue stubble prior to the next crop season. One example is a product sold under the trademark of Bio Reverse by The Andersons Inc. of Maumee, Ohio. Another microbial based biodegradation product is sold by a Hefty Seed Company of Baltic South Dakota under the trademark of D- Comp. Two other products are sold by Biodyne World, Inc. of Sarasota, Florida for breakdown of crop residue under the trademarks MELTDOWN and ENVIRON OC 501.

### SUMMARY

This disclosure describes a composition for degrading cellulosic material wherein the composition comprises a blend of water, ammonium sulfate, anti-foam and alkyl polysaccharide in a range of 10 to 30% alkyl polysaccharide in 0.1 to 3.4 lbs. of ammonium sulfate per 1 gallon of the blend and a bacteria spore blend or a blend of non-spore forming bacteria comprising bacteria suitable for degrading plant residue. In another embodiment, the composition comprises a blend of water, ammonium sulfate, anti-foam and alkyl polysaccharide in a range of 15 to 40% alkyl polysaccharide in 0.1 to 3.0 lbs. of ammonium sulfate per 1 gallon of the blend and a bacteria spore blend or a blend of non-spore forming bacteria comprising bacteria suitable for degrading plant residue. In yet another embodiment, the composition comprises a blend of water, ammonium sulfate, anti-foam and alkyl polysaccharide in a range of 20 to 55% alkyl polysaccharide in 0.1 to 2.5 lbs. of ammonium sulfate per 1 gallon of the blend and a bacteria spore blend or a blend of non-spore forming bacteria comprising bacteria suitable for degrading plant residue.

Furthermore, the bacteria may comprise either or both a bacteria spore blend or non-spore forming bacteria.

Furthermore, the bacillus spore blend comprises spores of Bacillus coagulans, Bacillus subtils, Bacillus pumilis, Bacillus megaterium and Bacillus amyloliquefaciens.

Furthermore, the composition is applied to plant residue.

Furthermore, the Bacillus coagulans, Bacillus subtils, Bacillus pumilis, Bacillus megaterium and Bacillus amyloliquefaciens are each at approximately a concentration of 3×10⁷ CFUs per gram.

Furthermore, the bacteria spore blend or the non-spore forming bacteria develops into bacteria sufficient to breakdown plant residue thereby increasing total nitrogen, phosphate and potassium soil content when compared to untreated soil.

This disclosure also includes a method of applying a composition on plant residue wherein the composition comprises a blend of water, ammonium sulfate, anti-foam and alkyl polysaccharide in a range of 10 to 30% alkyl polysaccharide in 0.1 to 3.4 lbs. of ammonium sulfate per 1 gallon of the blend and a bacteria spore blend or a blend of non-spore forming bacteria comprising bacteria suitable for degrading plant residue. In another embodiment, the composition comprises a blend of water, ammonium sulfate, anti-foam and alkyl polysaccharide in a range of 15 to 40% alkyl polysaccharide in 0.1 to 3.0 lbs. of ammonium sulfate per 1 gallon of the blend and a bacteria spore blend or a blend of non-spore forming bacteria comprising bacteria suitable for degrading plant residue. In yet another embodiment, the composition comprises a blend of water, ammonium sulfate, anti-foam and alkyl polysaccharide in a range of 20 to 55% alkyl polysaccharide in 0.1 to 2.5 lbs. of ammonium sulfate per 1 gallon of the blend and a bacteria spore blend or a blend of non-spore forming bacteria comprising bacteria suitable for degrading plant residue.

Furthermore, this method includes that the bacillus blend of spores comprises spores of Bacillus coagulans, Bacillus subtils, Bacillus pumilis, Bacillus megaterium and Bacillus amyloliquefaciens.

Furthermore, this method includes that the Bacillus coagulans, Bacillus subtils, Bacillus pumilis, Bacillus megaterium and Bacillus amyloliquefaciens are each at approximately a concentration of 3×10⁷ CFUs per gram.

### DETAILED DESCRIPTION

This disclosure includes a composition for application in the field to break down crop residue left in the field after harvest or residue from cover crops thereby improving planter performance and releasing nutrients back into the soil. The composition may also be used to break down any type of plant reside or residue containing plant material such as manure. The composition is most beneficial in reduced tillage situations wherein crop residue remains lying on the field after harvest and will not be plowed under. Such crop residue obstructs consistent and selected seed planting. The composition is also useful for reducing thatch in grass lawns, both residential and commercial settings. For purposes of this patent application, the phrase “plant residue” shall mean crop residue or plant stubble left in the field after harvest or otherwise, residue from cover crops, thatch, manure, or other cellulosic containing material.

When applied to the soil after the crop has been harvested which is the most common situation, the composition of this disclosure breaks down stubble and all crop residue left after the crop has been harvested. The composition breaks down plant stubble or plant residue to an extent such that seeds may be planted in the planting furrow at a consistent selected depth and at a consistent selected seed spacing. Selected seed depth may vary with plant species, for example corn and soybean seeds are planted at different depths. Soil to seed contact is improved since the previous harvest's stubble and other residue has been broken down and eliminated. Soil type and moisture are also factors for consideration in seed depth. Planting seeds at the selected optimal depth consistently greatly improves seed germination and therefore yield. In addition, if stubble or other crop residue obstructs placement of a seed into the soil, then a nonproductive space occurs within a seed row. If such a nonproductive space occurs frequently, yields can be affected considerably.

It has also been found that the composition of this disclosure if used with glyphosate increases the herbicidal effect of glyphosate. It appears that the composition of this disclosure with its bacteria spore blend or a blend of non-spore forming bacteria of this disclosure as discussed below not only breaks down crop residue, it also has a synergistic effect on glyphosate for controlling weeds. Glyphosate or other herbicides are typically applied in the spring but may also be applied in the fall after harvest to control weeds that may be emerging. Thus, the use of the composition of this disclosure with its bacteria spore blend or a blend of non-spore forming bacteria not only reduces plant stubble but also provides greater effective weed control.

The composition of this disclosure includes a composition for application on plant stubble or plant residue left on a field after harvest wherein the composition comprises a blend of water, ammonium sulfate, anti-foam and alkyl polysaccharide in a range of 10 to 30% alkyl polysaccharide in 0.1 to 3.4 lbs. of ammonium sulfate per 1 gallon of the blend and a bacteria spore blend or a blend of non-spore forming bacteria comprising bacteria suitable for degrading plant residue. In another embodiment, the composition comprises a blend of water, ammonium sulfate, anti-foam and alkyl polysaccharide in a range of 15 to 40% alkyl polysaccharide in 0.1 to 3.0 lbs. of ammonium sulfate per 1 gallon of the blend and a bacteria spore blend or a blend of non-spore forming bacteria comprising bacteria suitable for degrading plant residue. In yet another embodiment, the composition comprises a blend of water, ammonium sulfate, anti-foam and alkyl polysaccharide in a range of 20 to 55% alkyl polysaccharide in 0.1 to 2.5 lbs. of ammonium sulfate per 1 gallon of the blend and a bacteria spore blend or a blend of non-spore forming bacteria comprising bacteria suitable for degrading plant residue.

To aid the selected blend of bacteria into attaching to the stubble and crop residue, a surfactant such as alkyl polysaccharide is used. The surfactant of this disclosure greatly enhances the bacterial function by acting as a coating thereby providing a method of attaching the bacterial spores to the stubble and crop residue and also providing a carbohydrate source for the bacillus bacteria to germinate from dormancy into a reproductive stage and then to multiply to sufficient numbers to breakdown the stubble and crop residue as described herein.

The selected blend of bacteria spores when applied to the field reverts to bacteria which then multiply sufficiently to stimulate and fortify biological activity that breaks down the stubble and other crop residue, releasing available nutrients directly into the field.

To greatly aid the selected blend of bacteria into attaching to the stubble and crop residue, a surfactant such as an alkyl polysaccharide is used. The surfactant of this disclosure greatly enhances the bacterial function by acting as a coating in relation to the stubble or crop residue thereby providing a method of attaching the bacterial spores to the stubble and crop residue and also providing a carbohydrate source for the bacillus bacteria to germinate from dormancy into a reproductive stage and then to multiply to sufficient numbers to breakdown the stubble and crop residue as described herein.

A surfactant improves wettability and penetration of the spray solution when in contact with crop residue. This results in an increase in permeation of the microbes into the crop residue significantly increasing performance and the viability of microbes. A surfactant is a compound that reduces interfacial tensions between two liquids or between a liquid and a solid. On a molecular level, surfactants are typically molecules containing one portion that is polar and one portion that is non-polar. The polar, water-soluble portion is sometimes referred to as hydrophilic (“water loving”) while the non-polar, water-insoluble portion is sometimes referred to as hydrophobic (“water hating”) or lipophilic (“fat loving”). Surfactants are sometimes referred to as amphiphilic because of their dual character.

Suitable surfactants are soluble in water, have a hydrophilic-lipophilic balance (HLB) greater than about 10, are environmentally friendly (relatively low in toxicity to plants and animals) and are non-toxic to microbes and microbial spores.

In one example, an alkyl polysaccharide sold under the name, Agrimule PG 2069®, APG 325®, manufactured by BASF Corporation is contemplated. Agrimule PG 2069® includes alkyl polyglycoside polymers with alkyl chains of nine carbon atoms in a concentration of 20% by weight, ten carbon atoms in a concentration of 40% by weight and eleven carbon atoms in a concentration of 40% by weight. The alkyl polyglycoside has an average degree of polymerization of 1.6. The alkyl polyglycoside is a nonionic surfactant. The alkyl polyglycoside is non-gelling, biodegradable and soluble in dispersions of high salt concentrations.

One other alkyl polysaccharide supplier is Croda International plc of East Yorkshire, United Kingdom. The alkyl polysaccharide manufactured by Croda is sold under the trade name AT PLUS 452®. This alkyl polysaccharide is based on glucose and fatty alcohols derived from plant sources.

Other suitable non-ionic type surfactants are betaine surfactants sold under the trade name Amphosol DMX manufactured by Stepan Northbrook, IL. Solvay, Brussels Belgium also manufactures suitable non-ionic surfactants.

The “solution” has a pH of about from 0 to 5, preferably between 1.5 to 4 with buffering capacity between the pH values of 2 to 4.5. The solution should include adequate buffering agents to maintain a pH between 1.5 to 4 upon diluting in water at ratios as between 1:5 to as high as 1:200.

The desired pH in the “solution” is achieved by including suitable buffers. Suitable buffers will have at least one function group with a pKa value between 2.5 to 5. A pKa value is defined as the negative of the logarithm of the equilibrium constant Ka for the reaction

HA [symbol] H++A-

i.e.,

Ka=[H+][A-]/[HA]

where [H+], etc. represent the concentrations of the respective species in mol/L. It follows that pKa=pH+log[HA]−log[A-], so that a solution with 50% dissociation has pH equal to the pKa of the acid.

Suitable buffers include but are not limited to amino acids, carboxylic acids, phosphoric acids and their derivatives, phosphonic acids and their derivatives, sulfonic acids and their derivatives.

Preferred buffering agents are relatively low in cost, readily available, and environmentally friendly (relatively low in toxicity to plants and animals). Preferred buffering agents include lactic acid, citric acid, fulvic acid, lignin-sulfonic acid, phosphorus acid, phosphoric acid, acetic acid, malic acid, citric acid, glycolic acid, gluconic acid, glucoheptonic acid, fulvic acid, humic acid, and salicylic acid.

The rheology modifying agent and/or suspending agents in the composition of this disclosure function preferably together to maintain the bacillus spores in suspension, and to make the bacillus spores resistant to agglomeration. For purposes of this application the phrase “rheology modifying agent and/or suspending agent(s)” will be used to refer to one or several agents as described herein to provide the function of suspending the bacillus spores.

Agglomeration of spores increases their effective particle size, making it more difficult to remain in suspension. The rheology modifying agent and/or suspending agents can function to suspend bacillus spore particles through the mechanism of producing high yield-stress fluids that have limited effect on viscosity, or through the mechanism of viscosity.

The rheology modifying agent can either produce high performance at low viscosity or can produce a high viscosity fluid that has high shear thinning properties and is very pseudo- plastic or thixotropic. The suspending agents can be nonionic, cationic or anionic.

Suitable anionic suspending and/or rheology modifying agents include without limitation xanthan gums, guar gums, acrylate copolymers, alkali swellable emulsions (ASE) and (maleic anhydride decadiene) cross polymer.

Suitable nonionic suspending agents include without limitation hydrophobically modified polyurethanes (HEUR), hydrophobically modified polyethers (HPME), cellulose ethers, such as carboxymethyl cellulose, hydroxyethylcellulose, hydroxypropyl cellulose, hydroxypropylmethyl cellulose, and methylcellulose, microfibrillated cellulose, chemically modified cellulose.

Suitable inorganic suspending agents include without limitation organoclays, such as attapulgite, and fumed silicas.

Suitable suspending agents include without limitation lignin sulfonates, naphthalenesulfonate formaldehyde condensates, EO-PO-EO block copolymers, EO-PO block copolymers, tristyrylphenol ethoxylates, phosphate esters, acrylic graft copolymers, styrene acrylic copolymers.

Combinations of the foregoing suspending agents and/or rheology modifying agents can also be employed.

The suspending and/or rheology modifying agent may constitute about 0.01-5.0 percent by weight of the composition concentrated product. The selected effective amount of the suspending and/or rheology modifying agent depends largely on the activity of the suspending agent.

Fulvic acid in the form of Potassium fulvate is part of the composition of this disclosure at an approximate level of approximately 1.00 percent by weight of the composition. A suitable effective amount of fulvic acid is approximately 0.5 to 1.5 percent by weight of the composition.

The composition of this disclosure includes a consortium of bacillus bacteria that have been originally derived from the soil. In one embodiment, the consortium of bacillus bacteria includes the following, all being at a concentration of 3×10⁷ CFUs per gram. The bacteria in the consortium work together to breakdown cellulose and other structural components of plant residue. The consortium of bacillus bacteria includes the following along with the function of each bacillus bacteria in this consortium:

Bacillus licheniformis: Bacillus licheniformis produces enzymes that break the chemical bonds between carbon molecules in the stubble and crop residue.

Bacillus coagulans: In this bacterial consortium bacillus coagulans controls the production of lactic acid and the pH of the solution that the microbes swim in. Microbes in the presence of bacillus coagulans are able to be more effective breaking down cellulose and less soluble complex carbon structures.

Bacillus subtills: Bacillus subtils provides protection from pathogens and predatory microbes that consume other microbes in the consortium.

Bacillus pumilis: Bacillus pumilis releases phosphate and potassium from the stubble, crop residue and soil.

Bacillus megaterium: Bacillus megaterium produces enzymes that solubilize and breakdown carbon bonds in the stubble and crop residue.

Bacillus amyloliquefaciens: Bacillus amyloliquefaciens produces enzymes that solubilize and breakdown carbon bonds in the stubble and crop residue. The enzymes produced can breakdown tough cellulose.

The consortium of bacillus bacteria of the composition of this disclosure breaks down the plant cells. As the plant cells are broken down the cells leak nitrogen, potassium, phosphate, zinc, manganese, copper, sulfur, and other nutrients. The amount of nutrients released over a given amount of time depends on the temperature, nutrient content of the stubble and crop residue, and the amount of nitrogen present to feed the reaction.

In general, it is believed that the amount of phosphate released by the composition of this disclosure was between about five and ten times higher than control levels. Potassium may be released about between 50 percent and 100 percent higher than the release of the control. It has also been found that form of potassium released by corn stubble was about 3 times more available to plants than the potassium found in regular potash applied as fertilizer. Nitrogen was up to about 5 times higher in the leachate from corn stubble treated with the composition of this disclosure.

The following table shows results of one test using the composition of this disclosure on corn stubble and residue, testing the soil for the nutrients indicated.

TABLE

| | Nutrient | | | | |
|---|---|---|---|---|---|
| SAMPLE | Total | | | | |
| ID | Nitrogen | NO₃ | NH₄ | Phosphate | Potassium |
| 15 days UT | 44.78 | 36.74 | 8.04 | 3.37 | 216.31 |
| 15 days TR | 275.78 | 34.86 | 240.92 | 116.57 | 322.46 |
| 30 days UT | 53.89 | 45.99 | 7.90 | 5.36 | 178.91 |
| 30 days TR | 259.85 | 16.71 | 243.14 | 270.20 | 564.79 |

Values in the above table are in parts per million.

Samples of the soil at a depth of 0 to 12 inches were taken 15 days after treatment with the composition of this disclosure (TR) applied and a sample of the soil untreated (UT). Samples were also taken 30 days after treatment both of treated soil and untreated soil. As the table indicates total nitrogen, NH₄, phosphate and potassium levels greatly increased in the soil both 15 days and 30 days after treatment. Total nitrogen was increased 6-fold, ammonium (NH₄) was increased 30-fold, phosphate was increased 34-fold and potassium was increased 1.5-fold after 15 days when compared to untreated soil. While after 30 days of treatment, total nitrogen was increased more than 4-fold, ammonium (NH4) was increased 31-fold, phosphate was increased 50-fold and potassium was increased 3-fold when compared to untreated soil.

What is claimed is:

1. A composition for application on plant stubble or plant residue left on a field after harvest, the composition comprising:
a composition for application on plant stubble or plant residue left on a field after harvest wherein the composition comprises a blend of water, ammonium sulfate, anti-foam and alkyl polysaccharide comprises an approximate range of 10 to 30% alkyl polysaccharide in 0.1 to 3.4 lbs. of ammonium sulfate per 1 gallon of the blend and a bacteria spore blend or a blend of non-spore forming bacteria comprising bacteria suitable for degrading plant residue.

2. The composition of claim 1 wherein the blend of water, ammonium sulfate, anti-foam and alkyl polysaccharide comprises an approximate range of 15 to 40% alkyl polysaccharide in 0.1 to 3.0 lbs. of ammonium sulfate per 1 gallon of the blend.

3. The composition of claim 1 wherein the blend of water, ammonium sulfate, anti-foam and alkyl polysaccharide comprises an approximate range of 20 to 55% alkyl polysaccharide in 0.1 to 2.5 lbs. of ammonium sulfate per 1 gallon of the blend.

4. The composition of claim 1 where in the bacteria spore blend comprises spores of Bacillus coagulans, Bacillus subtils, Bacillus pumilis, Bacillus megaterium and Bacillus amyloliquefaciens.

5. The composition of claim 1 wherein the Bacillus coagulans, Bacillus subtils, Bacillus pumilis, Bacillus megaterium and Bacillus amyloliquefaciens are each at approximately a concentration of 3×10⁷ CFUs per gram.

6. The composition of claim 1 wherein the bacteria spore blend develops into a bacteria blend sufficient to breakdown the plant stubble or plant residue thereby increasing total nitrogen, phosphate and potassium soil content when compared to untreated soil.

7. A method for reducing plant stubble or plant residue left on the field after harvest and increasing nutrients in the soil from the breakdown of the plant stubble or plant residue, the method comprising:
applying a composition on plant stubble or plant residue left on a field after harvest wherein the composition comprises a blend of water, ammonium sulfate, anti-foam and alkyl polysaccharide comprises an approximate range of 10 to 30% alkyl polysaccharide in 0.1 to 3.4 lbs. of ammonium sulfate per 1 gallon of the blend and a bacteria spore blend or a blend of non-spore forming bacteria comprising bacteria suitable for degrading plant residue.

8. The method of claim 7 wherein the approximate range comprises 15 to 40% alkyl polysaccharide in 0.1 to 3.0 lbs. of ammonium sulfate per 1 gallon of the blend.

9. The method of claim 7 wherein the approximate range comprises 20 to 55% alkyl polysaccharide in 0.1 to 2.5 lbs. of ammonium sulfate per 1 gallon of the blend.

10. The method of claim 5 wherein the bacteria spore blend comprises spores of Bacillus coagulans, Bacillus subtils, Bacillus pumilis, Bacillus megaterium and Bacillus amyloliquefaciens.

11. The method of claim 6 wherein the Bacillus coagulans, Bacillus subtils, Bacillus pumilis, Bacillus megaterium and Bacillus amyloliquefaciens are each at approximately a concentration of 3×10⁷ CFUs per gram.

12. The method of claim 5 wherein the bacteria spore blend develops into a bacteria blend sufficient to breakdown the plant stubble or plant residue thereby increasing total nitrogen, phosphate and potassium soil content when compared to untreated soil.
